(19) United States
(12) Patent Application Publication
(10) Pub. No.: US 2016/0100972 A1
(43) Pub. Date: Apr. 14, 2016

(54) Therapeutic Band

(71) Applicant: Judy Stephenson, Jacksonville, NC (US)

(72) Inventor: Judy Stephenson, Jacksonville, NC (US)

(21) Appl. No.: 14/877,561

(22) Filed: Oct. 7, 2015

Related U.S. Application Data
(60) Provisional application No. 62/063,739, filed on Oct. 14, 2014.

(51) Int. Cl. A61F 5/01; A61F 5/058

(52) U.S. Cl. CPC A61F 5/013; A61F 5/05866

(57) ABSTRACT

A therapeutic band including an elastic band having an upper portion for engaging across a base of a wearer's index and middle fingers at a front surface of the wearer's hand; a lower portion for engaging across a top portion of a wearer's wrist; and two opposing side portions that extends between the upper portion and the lower portion. The therapeutic band may further include an adjustable bridge portion, wherein when worn the adjustable bridge portion traverses across a palm of the wearer's hand and adjustably connects between the two opposing side portions.

### RELATED APPLICATIONS

This application is related to and claims priority to U.S. Provisional Patent Application No. 62/063,739, filed on Oct. 14, 2014, entitled “Therapeutic Band”; the entire disclosure of which is incorporated herein by reference.

### FIELD OF THE INVENTION

The invention generally relates to a therapeutic band. In particular, the invention is directed to a carpel tunnel therapeutic band.

### BACKGROUND OF THE INVENTION

Most of the small muscles that work the thumb and pinky finger start on the carpal bones. These muscles connect in ways that allow the hand to grip and to hold. The thenar muscles of the hand allow the thumb to move across the palm of the hand, an important function called thumb opposition. Carpal tunnel syndrome (CTS) is a condition brought on by increased pressure on the median nerve at the wrist. In effect, it is a pinched nerve at the wrist. Symptoms may include numbness, tingling, and pain in the arm, hand and fingers. The originating cause of this condition is unknown. However, the constriction of the carpal tunnel results in increased pressure on the median nerve, resulting in weakness, numbness, and pain in the hand.

There is a need for a therapeutic band that provides relief from the discomfort caused by CTS and that aids in exercising weakened muscles and tendons of the hand and/or wrist.

There is also a need for a therapeutic band that allows the thenar muscles to be lifted and supported by way of reciprocal pressure between these muscles and the extensor tendons on top of the hand to help improve control and flexibility and to also decreased pressure on the median nerve, allowing it to remain unrestricted in the carpal tunnel to carry out its function of feeling and movement in the hand.

### BRIEF DESCRIPTION OF THE INVENTION

In one embodiment the invention provides a therapeutic band, including an elastic band and an adjustable bridge portion, wherein when worn the adjustable bridge portion traverses across a palm of the wearer's hand and adjustably connects between the two opposing side portions. The elastic band may further include an upper portion for engaging across a base of a wearer's index and middle fingers at a front surface of the wearer's hand; a lower portion for engaging across a top portion of a wearer's wrist; and two opposing side portions that extend between the upper portion and the lower portion. The elastic band may be generally circular in shape. One of the two opposing side portions may extend from a first point of the lower portion that is proximal to a base of a wearer's thumb and then wraps under the base of the wearer's thumb extending to a first point of the upper portion, and wherein the other of the two opposing side portions may extend from a second point of the upper portion that is proximal to a space between a wearer's middle and ring fingers and may extend in a generally downward direction traversing an underside of the wearer's ring and pinky fingers to a second point of the lower portion. The first point and second point of the lower portion are generally located in a region of a top side of a wearer's wrist and spaced apart from one another, such that the first point of the lower portion is in a region of the wearer's wrist substantially aligned with the base of the wearer's thumb and the second point of the lower portion is in a region of the wearer's wrist substantially aligned with the wearer's pinky finger. The first point and second point of the upper portion may be spaced apart such that the first point of the upper portion is in a region below a base of the wearer's index finger and the second point of the upper portion is located in a region between the wearer's middle and ring fingers. The lower portion may be of a substantially constant width; the opposing side portion extending from the first point of the lower portion to the first point of the upper portion may narrow in width as it extends; the top portion may continue to narrow as it crosses over the base of the wearer's index and middle fingers at the front surface of the wearer's hand; and the other opposing side portion may be a substantially constant width and narrower than that of the lower portion, other opposing side portion, and upper portion in the region between the wearer's middle and ring fingers, and may widen to a width substantially the same as that of the lower portion as it extends downward across the underside of the wearer's ring and pinky fingers to the second point of the lower portion. The opposing side portion that extends from the second point of the upper portion to the second point of the lower portion may extend at an angle in the range of about 45 degrees relative to the lower portion. The adjustable bridge adjustably connects the two opposing side portions across the palm of the wearer's hand. The adjustable bridge may be moveably attached to one of the two opposing side portions and may be releasably attached to the other of the two opposing side portions. Alternatively, the adjustable bridge may be fixed to one of the two opposing side portions and releasably attached to the other of the two opposing side portions. The adjustable bridge may be releasably attached to one of the two opposing side portions by any one of a hook and loop fastener, snap, button, clip, buckle, tie/lace, and clasp. The therapeutic band may further include a protective sleeve engaged around a portion of the elastic band in the region disposed between a wearer's middle and ring fingers.

These and other embodiments will be apparent from the ensuing specification.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 illustrates a bottom perspective view of a therapeutic band in accordance with an embodiment of the invention;

FIG. 2 illustrates a top plan view of the therapeutic band in accordance with an embodiment of the invention;

FIG. 3 illustrates a bottom plan view of the therapeutic band in accordance with an embodiment of the invention;

FIG. 4 illustrates a right side elevation view of the therapeutic band in accordance with an embodiment of the invention; and

FIG. 5 illustrates a left side elevation view of the therapeutic band in accordance with an embodiment of the invention.

### DETAILED DESCRIPTION OF THE INVENTION

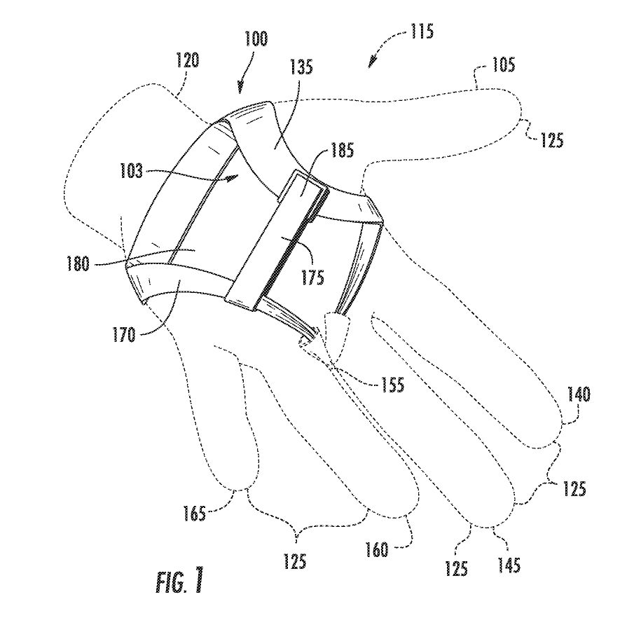

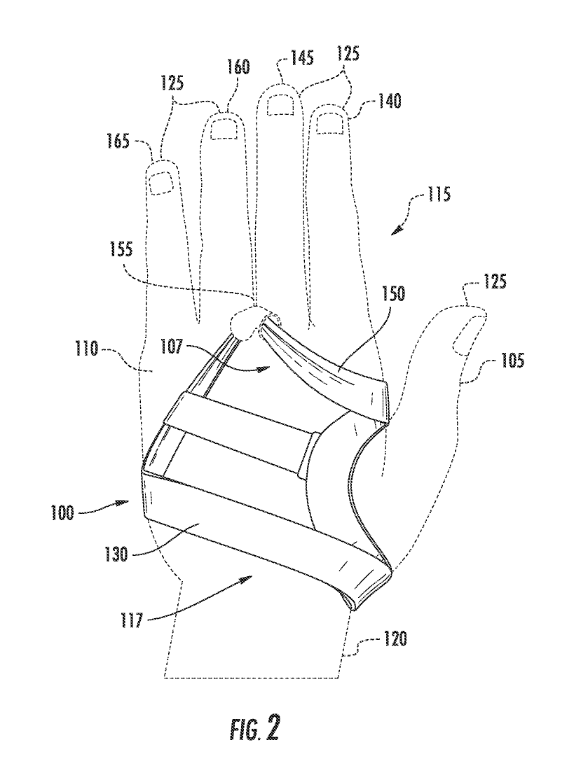

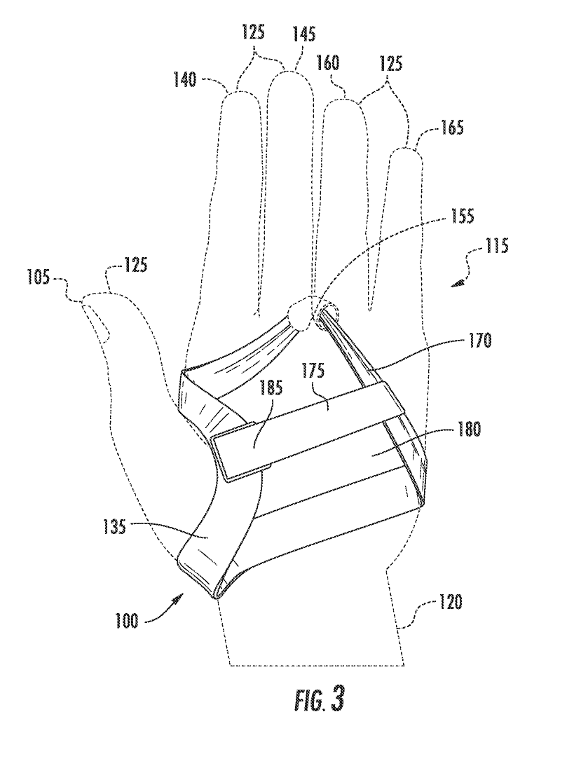

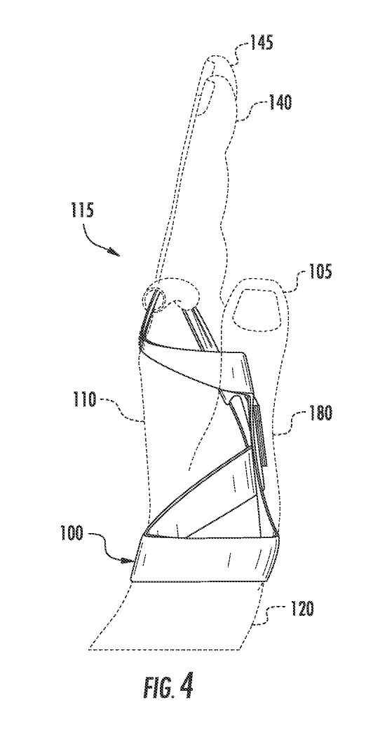

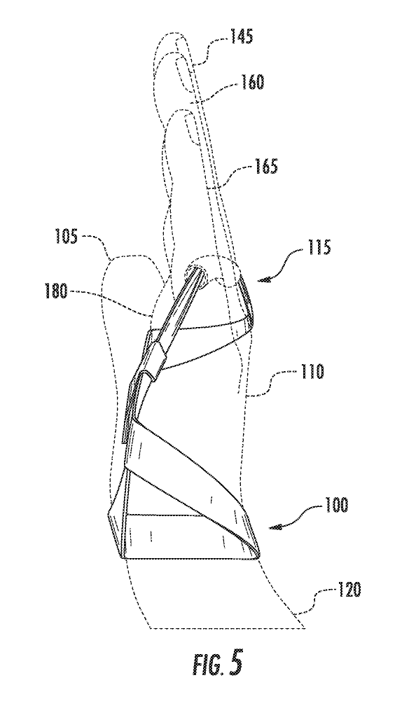

Reference is now made to FIG. 1-5, wherein like reference numerals are used to refer to like elements throughout. In the following description, for purposes of explanation, numerous specific details are set forth in order to provide a thorough understanding thereof It may be evident, however, that the novel embodiments can be practiced without these specific details. In other instances, well known structures and devices are shown in block diagram form in order to facilitate a description thereof.

Therapeutic band 100 provides a band that follows the natural, lower contour of the thenar muscles (located generally in the area indicated by arrow 103) that make up the thumb 105, crossing the extensor tendons (located generally in the area shown by arrow 107) on the upper surface (front of hand) 110 of a user's hand 115, which perform like “lifters” in flexing and extending the metacarpals. Therapeutic band 100 allows the thenar muscles to be lifted and supported by way of reciprocal pressure between these muscles and the extensor tendons on top of the hand 110. This results in improved control and flexibility and decreased pressure on the median nerve (located generally in the area indicated by arrow 117), allowing it to remain unrestricted in a person's carpal tunnel to carry out its function of feeling and movement in the hand 115. The therapeutic band 100 acts to both support and exercise the thenar muscles of the thumb 105 and the extensor tendons of the hand 115, adding control and mobility in a wearer's wrist 120 and fingers 125 for fine motor control.

Therapeutic band 100 preferably includes a section 130 that crosses over the extensor compartments across a top portion of wrist 120, then wraps under thumb 105 along a section 135 to support the thenar border where it then rises to an upper portion of the base of thumb 105 (i.e., approximately where the base of thumb 105 and pointer finger 140 meet). Therapeutic band 100 then crosses over a portion of the front of hand 110, and thus the extensor digital muscles of the index 140 and middle fingers 145, along a section 150, traveling through a space 155 between the middle 145 and ring finger 160, and then extending in a generally downward direction across the underside of ring finger 160 and pinky finger 165 along a section 170. Section 170 may descend at an angle in the range of about 45 degrees relative to section 130.

An adjustable bridge 175 may connect opposing sides of therapeutic band 100 (i.e., extending between approximately the midsection of sections 135 and 170) across the palm of hand 180. Adjustable bridge 175 allows for a user to adjust the fit of therapeutic band 100 according the user's hand size and support need.

In one embodiment, therapeutic band 100 may be made of an elastic material, which may have a substantially flat thickness. Therapeutic band 100 may be generally circular circular in shape, and may sections of varying width based on what portion of the hand they correspond to. In one example, therapeutic band 100 may vary in width from in the range of about ½ inch as it crosses the upper wrist area (i.e., section 130) of the hand 115 and under the thenar muscles of the thumb (i.e., section 135), traveling over the extensor tendons at the base of the index and middle fingers (i.e., section 150) and narrowing to in the range of about ⅛ inch as it passes through the space 120 between the middle 145 and ring finger 160 and then widening to in the range of about ½ inch as it passes underneath both middle 145 and ring finger 160 (i.e., section 170) and then connecting with the upper side of the wrist.

Adjustable bridge band 175 may connect opposing sections of therapeutic band 100 (i.e., section 170 and 135) across the user's palm 180 to allow for increased/decreased pressure and comfort by adjustment mechanism 185. Adjustment mechanism 185 may be any number of suitable mechanisms, including but not limited to, hook and loop type fastener, snap, clip, buckle, button, clasp, tie/lace, or any other suitable adjustment mechanism. In one example, adjustable bridge band 175, at one end, engages one of sections 170 and 135 such that it may be positionable, e.g., slidable, along at least a portion of the length of the respective section 170 or 135; and adjustable bridge band 175 is adjustably attached at its other end to the other of sections 170 and 135 by adjustment mechanism 185. In another example, adjustable bridge band 175 at one end is permanently fixed to one of sections 170 and 135, and adjustably attached at its other end to the other of sections 170 and 135 by adjustment mechanism 185.

The design of the therapeutic band 100 allows the wearer to place it comfortably over and under the surface of his/her hand 110 and palm 180, which requires the greatest support during movement to include lifting, grasping and flexing of the fingers 125. The wearer can comfortably adjust the pressure of the therapeutic band 100 by taking up or letting out the bridging band 175 across the palm 180 of hand 110 via adjustment mechanism 185.

Therapeutic band 100 may provide a number of therapeutic functions. The circular, elastic design allows therapeutic band 100, when worn, to follow the natural contour of the wearer's hand 115 to provide a natural support, without restricting the movement and use of the wearer's hand 115 and fingers 125. In addition, the therapeutic band 100 supports weakened muscles and tendons and thus allows them to engage and strengthen while carrying out tasks that are essential to the natural movement of the wearer's hand 115 and fingers 125.

The therapeutic band 100 supports the critical thenar muscles and extensor tendons in their work to assist the fingers 125 and hand 115 in flexing, supporting and grasping, which allows for strengthening, aligning and mobilizing the underlying muscles. The therapeutic band 100 may be used periodically, intermittently, or as-needed. Periodic use of the therapeutic band 100 allows for a natural coordination of muscles and tendons, producing a counterbalance of movement in the hand 115 and fingers 125. Intermittent use of the therapeutic band 100 provides supportive flection of the thenar muscles which, in turn, reduces the pain associated with the narrowing of the carpal tunnel found in carpal tunnel syndrome. As-needed use of the therapeutic band 100 allows for unrestricted movement of the fingers 125, tendons and muscles of the hand 115 to be employed throughout the day, providing comfort, support, and strengthening of the affected muscles and tendons.

The therapeutic band 100 allows for improved alignment, control, and flexibility that are needed for proper functioning of the wrist 120, hand 115, and fingers 125 while working or using the hand 115. Used periodically during the day, therapeutic band 100 can provide comfortable support while exercising the weakened muscles and tendons that contribute to the narrowing of the carpal tunnel.

The foregoing detailed description of embodiments refers to the accompanying drawings, which illustrate specific embodiments of the invention. Other embodiments having different structures and operations do not depart from the scope of the present invention. The term “the invention” or the like is used with reference to specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

Following long-standing patent law convention, the terms “a,” “an,” and “the” refer to “one or more” when used in this application. Thus, for example, reference to “a subject” includes a plurality of subjects, unless the context clearly is to the contrary (e.g., a plurality of subjects), and so forth.

Throughout this specification, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

What is claimed is:

1. A therapeutic band, comprising:
a. an elastic band comprising:
i. an upper portion for engaging across a base of a wearer's index and middle fingers at a front surface of the wearer's hand;
ii. a lower portion for engaging across a top portion of a wearer's wrist; and
iii. two opposing side portions that extend between the upper portion and the lower portion; and
b. an adjustable bridge portion, wherein when worn the adjustable bridge portion traverses across a palm of the wearer's hand and adjustably connects between the two opposing side portions.

2. The therapeutic band of claim 1, wherein the elastic band is generally circular in shape.

3. The therapeutic band of claim 1, wherein one of the two opposing side portions extends from a first point of the lower portion that is proximal to a base of a wearer's thumb and then wraps under the base of the wearer's thumb extending to a first point of the upper portion, and wherein the other of the two opposing side portions extends from a second point of the upper portion that is proximal to a space between a wearer's middle and ring fingers and extends in a generally downward direction traversing an underside of the wearer's ring and pinky fingers to a second point of the lower portion.

4. The therapeutic band of claim 3, wherein the first point and second point of the lower portion are generally located in a region of a top side of a wearer's wrist and spaced apart from one another, such that the first point of the lower portion is in a region of the wearer's wrist substantially aligned with the base of the wearer's thumb and the second point of the lower portion is in a region of the wearer's wrist substantially aligned with the wearer's pinky finger.

5. The therapeutic band of claim 3, wherein the first point and second point of the upper portion are spaced apart such that the first point of the upper portion is in a region below a base of the wearer's index finger and the second point of the upper portion is located in a region between the wearer's middle and ring fingers.

6. The therapeutic band of claim 3, wherein, the lower portion is of a substantially constant width; the opposing side portion extending from the first point of the lower portion to the first point of the upper portion narrows in width as it extends; the top portion continues to narrow as it crosses over the base of the wearer's index and middle fingers at the front surface of the wearer's hand; and the other opposing side portion is a substantially constant width and narrower than that of the lower portion, other opposing side portion, and upper portion in the region between the wearer's middle and ring fingers, and widens to a width substantially the same as that of the lower portion as it extends downward across the underside of the wearer's ring and pinky fingers to the second point of the lower portion.

7. The therapeutic band of claim 1, wherein the opposing side portion that extends from the second point of the upper portion to the second point of the lower portion extends at an angle in the range of about 45 degrees relative to the lower portion.

8. The therapeutic band of claim 1, wherein the adjustable bridge adjustably connects the two opposing side portions across the palm of the wearer's hand.

9. The therapeutic band of claim 8, wherein the adjustable bridge is moveably attached to one of the two opposing side portions and releasably attached to the other of the two opposing side portions.

10. The therapeutic band of claim 8, wherein the adjustable bridge is fixed to one of the two opposing side portions and releasably attached to the other of the two opposing side portions.

11. The therapeutic band of claim 8, wherein the adjustable bridge is releasably attached to one of the two opposing side portions by one of a hook and loop fastener, snap, button, clip, buckle, tie/lace, and clasp.

12. The therapeutic band of claim 3, further comprising a protective sleeve engaged around a portion of the elastic band in the region disposed between a wearer's middle and ring fingers.
